(19) United States
(12) Patent Application Publication
(10) Pub. No.: US 2008/0145839 A1
(43) Pub. Date: Jun. 19, 2008

(54) Method and Nucleic Acids For the Differentiation of Astrocytoma, Oligoastrocytoma and Oligodenroglioma Tumor Cells

(75) Inventors: Alexander Olek, Berlin (DE); Christian Piepenbrock, Berlin (DE); Kurt Berlin, Stahnsdorf (DE)

Correspondence Address: Davidson, Davidson & Kappel, LLC, 485 7th Avenue, 14th Floor, New York, NY, 10018, US

(73) Assignee: Epigenomics AG, Berlin (DE)

(21) Appl. No.: 10/311,506

(22) PCT Filed: Jul. 2, 2001
(86) PCT No.: PCT/EP01/07539

§ 371 Date: Dec. 16, 2002

(30) Foreign Application Priority Data
Jun. 30, 2000 (DE) 100 32 529.7
Sep. 1, 2000 (DE) 100 43 826.1

(51) Int. Cl. C12Q 1/68; C07H 21/04; C40B 40/06; C40B 50/14

(52) U.S. Cl. 435/6; 536/22.1; 536/24.3; 506/30; 506/16

(57) ABSTRACT

Chemically modified nucleic acid sequences of genomic DNA, oligonucleotides and/or PNA-oligomers for detecting the cytosine methylation state of genomic DNA. In addition, a method for ascertaining genetic and/or epigenetic parameters of genes for the characterization, classification, differentiation, grading, staging, treatment and diagnosis of oligodendrogliomas, astrocytomas and oligoastrocytomas.

### FIELD OF THE INVENTION

The levels of observation that have been studied by the methodological developments of recent years in molecular biology, are the genes themselves, the translation of these genes into RNA, and the resulting proteins. The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

The present invention relates to nucleic acids, oligonucleotides, PNA-oligomers and to a method for the characterization, classification, differentiation, grading, staging, treatment and diagnosis of oligodendrogliomas, astrocytomas and oligoastrocytomas, by analysis of the genetic and/or epigenetic parameters of genomic DNA, in particular with its cytosine methylation status.

### PRIOR ART

The incidence of brain tumors is 6 in 100,000, the majority of which are metastases from other types of cancers. Of the primary tumors, the most common are those arising in the glial cells, the gliomas. The most common of these include the astrocytomas and oligodendromas. Both arise from different forms of supportive brain tissue, the astrocytes and oligodendrocytes respectively.

These tumors may both develop from retained stem cells, therefore are often found in mixed gliomas, such as oligoastrocytomas. In the case of mixed gliomas, there is no consistent method of prediction of progression of tumors from low grade to anaplastic to malignant. The malignant progression is wholly dependant on the type of cell which predominates. For example, If the tumor is predominantly astrocytic, this could take only 6 months to 5 years. If it is predominantly oligodendroglial, malignant transformation of a “low grade” mixed glioma could occur within 3 to 10 years.

Initial diagnosis of gliomas is by scan imaging, (e.g. CT, MRI). This may be supplemented by histological and/or cytological analysis of biopsies. The use of molecular markers has been described, for example ‘Cyclooxygenase-2 expression in human gliomas: prognostic significance and molecular correlations.’ High-dimensional mRNA based approaches have recently been shown to be able to provide a better means to distinguish between different tumor types and benign and malignant lesions. Application as a routine diagnostic tool in a clinical environment is however impeded by the extreme instability of mRNA, the rapidly occurring expression changes following certain triggers (e.g. sample collection), and, most importantly, the large amount of mRNA needed for analysis (Lipshutz, R. J. et al., Nature Genetics 21:20-24, 1999; Bowtell, D. D. L. Nature genetics suppl. 21:25-32, 1999), which often cannot be obtained from aroutine biopsy. Shono et al. Cancer Res. 2001 Jun. 1; 61(11):4375-81.

Aberrant DNA methylation within CpG islands is common in human malignancies leading to abrogation or overexpression of a broad spectrum of genes (Jones, P. A. Cancer Res 65:2463-2467, 1996). Abnormal methylation has also been shown to occur in CpG rich regulatory elements in intronic and coding parts of genes for certain tumours (Chan, M. F., et al., Curr Top Microbiol Immunol 249:75-86, 2000). Highly characteristic DNA methylation patterns could also be shown for breast cancer cell lines (Huang, T. H.-M., et al., Hum Mol Genet. 8:459-470, 1999).

Abnormal methylation of genes has been linked to gliomas (e.g. Epigenetic silencing of PEG3 gene expression in human glioma cell lines. Maegawa et al. Mol. Carcinog. 2001 May; 31(1):1-9.). It has also been shown that methylation pattern analysis can be correlated with the development of low grade oligoastrocytomas (Aberrant methylation of genes in low-grade oligoastrocytomas. Costello J F, Plass C, Cavenee W K. Brain Tumor Pathol. 2000; 17(2):49-56). However, the techniques used in such studies (restriction landmark genomic scanning, imprinting analysis) are limited to research, they are unsuitable for use in a clinical or diagnostic setting, and do not provide the basis for the development of a medium or high throughput method for the analysis of gliomas.

5-methylcytosine is the most frequent covalent base modification in the DNA of eukaryotic cells. It plays a role, for example, in the regulation of transcription, in genetic imprinting, and in tumorigenesis. Therefore, the identification of 5-methylcytosine as a component of genetic information is of considerable interest. However, 5-methylcytosine positions cannot be identified by sequencing since 5-methylcytosine has the same base pairing behavior as cytosine. Moreover, the epigenetic information carried by 5-methylcytosine is completely lost during PCR amplification.

A relatively new and currently the most frequently used method for analyzing DNA for 5-methylcytosine is based upon the specific reaction of bisulfite with cytosine which, upon sub-sequent alkaline hydrolysis, is converted to uracil which corresponds to thymidine in its base pairing behavior. However, 5-methylcytosine remains unmodified under these conditions. Consequently, the original DNA is converted in such a manner that methylcytosine, which originally could not be distinguished from cytosine by its hybridization behavior, can now be detected as the only remaining cytosine using “normal” molecular biological techniques, for example, by amplification and hybridization or sequencing. All of these techniques are based on base pairing which can now be fully exploited. In terms of sensitivity, the prior art is defined by a method which encloses the DNA to be analyzed in an agarose matrix, thus pre-venting the diffusion and renatuation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. 1996 Dec. 15; 24(24):5064-6). Using this method, it is possible to analyze individual cells, which illustrates the potential of the method. However, currently only individual regions of a length of up to approximately 3000 base pairs are analyzed, a global analysis of cells for thousands of possible methylation events is not possible. However, this method cannot reliably analyze very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. 1997 Mar.-Apr.; 5(2):948) the bisulfite technique is only used in research. Always, however, short specific fragments of a known gene are amplified subsequent to a bisulfite treatment and either completely sequenced (Olek A, Walter J. The pre-implantation ontogeny of the H19 methylation imprint. Nat Genet. 1997 Nov.; 17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 Jun. 15; 25(12):2529-31, WO 95/00669) or by enzymatic digestion (Xiong Z, Laird P W. COBRA: a sensitive and quantitative. DNA methylation assay. Nucleic Acids Res. 1997 Jun. 15; 25(12):2532-4). In addition, detection by hybridization has also been described (Olek et al., WO 99/28498).

Further publications dealing with the use of the bisulfite technique for methylation detection in individual genes are: Grigg G, Clark S. Sequencing 5-methylcytosine residues in genomic DNA. Bioessays. 1994 Jun.; 16(6):431-6, 431; Zeschnigk M, Schmitz B, Dittrich B, Buiting K, Horsthemke B, Doerfiler W. Imprinted segments in the human genome: different DNA methylation patterns in the Prader-Willi/Angelman syndrome region as determined by the genomic sequencing method. Hum Mol. Genet. 1997 Mar.; 6(3):387-95; Feil R, Charlton J, Bird A P, Walter J, Reik W. Methylation analysis on individual chromosomes: improved protocol for bisulphite genomic sequencing. Nucleic Acids Res. 1994 Feb. 25; 22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19; 157(1-2):261-4; WO 97/46705, WO 95/15373 and WO 97/45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas M, Hillenkamp F. Laser desorption ionization of proteins with molecular masses exceeding 10,000 daltons. Anal Chem. 1988 Oct. 15; 60(20):2299-301). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionization Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionization process via the matrix is considerably less efficient. In MALDI-TOF spectrometry, the selection of the matrix plays an eminently important role. For the desorption of peptides, several very efficient matrixes have been found which produce a very fine crystallization. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. 1995 Apr. 25; 23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

### DESCRIPTION

The object of the present invention is to provide a means for the identification of brain tumor cells. More specifically, the present invention discloses a method and nucleic acids that enable the differentiation of the different cell types within oligoastrocytoma tumors. Identification of cell types is of great prognostic and therapeutic significance as prognosis is dependant upon the predominant cell type. Commonly used histological and cytological methods for such analysis require that tissue samples of an adequate size are available. The present invention is based on the discovery that genetic and epigenetic parameters, in particular, the cytosine methylation pattern of genomic DNA, are particularly suitable for the characterization, classification, differentiation, grading, staging, treatment and diagnosis of oligodendrogliomas, astrocytomas and oligoastrocytomas. Furthermore, the described invention enables the characterization, classification, differentiation, grading, staging, treatment and diagnosis of oligodendrogliomas, astrocytomas and oligoastrocytomas of cancer tissues using minute samples which would be inadequate for routine histological or cytological analysis.

This objective is achieved according to the present invention using a nucleic acid containing a sequence of at least 18 bases in length of the chemically pretreated genomic DNA according to one of Seq. ID No.1 through Seq. ID No.120.

The chemically modified nucleic acids (Seq. ID No.1 through Seq. ID No.120) could heretofore not be connected with the determination of genetic and epigenetic parameters.

The object of the present invention is further achieved by an oligonucleotide or oligomer for detecting the cytosine methylation state of chemically pretreated DNA, containing at least one base sequence having a length of at least 13 nucleotides which hybridizes to a chemically pretreated genomic DNA according to Seq. ID No.1 through Seq. ID No.120. The oligomer probes according to the present invention constitute important and effective tools which, for the first time, make it possible to determine the oligodendroglioma and/or oligoastrocytoma specific genetic and epigenetic parameters of chemically modified genomic DNA. The base sequence of the oligomers preferably contains at least one CpG dinucleotide. The probes may also exist in the form of a PNA (peptide nucleic acid) which has particularly preferred pairing properties. Particularly preferred are oligonucleotides according to the present invention in which the cytosine of the CpG dinucleotide is the 5^{th}-9^{th} nucleotide from the 5′-end of the 13-mer; in the case of PNA-oligomers, it is preferred for the cytosine of the CpG dinucleotide to be the 4^{th}-6^{th} nucleotide from the 5′-end of the 9-mer.

The oligomers according to the present invention are normally used in so called “sets” which contain at least one oligomer for each of the CpG dinucleotides of the sequences of Seq. ID No.1 through Seq. ID No.120. Preferred is a set which contains at least one oligomer for each of the CpG dinucleotides from one of Seq. ID No.1 through Seq. ID No.120.

Moreover, the present invention makes available a set of at least two oligonucleotides which can be used as so-called “primer oligonucleotides” for amplifying DNA sequences of one of Seq. ID No.1 through Seq. ID No.120, or segments thereof.

In the case of the sets of oligonucleotides according to the present invention, it is preferred that at least one oligonucleotide is bound to a solid phase. Moreover it is particularly preferred that all the oligonucleotides of one set are bound to the solid phase.

The present invention moreover relates to a set of at least 10 n (oligonucleotides and/or PNA-oligomers) used for detecting the cytosine methylation state in chemically pretreated genomic DNA (Seq. ID No.1 through Seq. ID No.120). These probes enable characterization, classification, differentiation, grading, staging, treatment and diagnosis of oligodendrogliomas, astrocytomas and oligoastrocytomas. The set of oligomers may also be used for detecting single nucleotide polymorphisms (SNPs) in chemically pretreated genomic DNA according to one of Seq. ID No.1 through Seq. ID No.120.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterized in that it is arranged on the solid phase in the form of a rectangular or hexagonal lattice. The solid phase surface is preferably composed of silicon, glass, polystyrene, aluminum, steel, iron, copper, nickel, silver, or gold. However, nitrocellulose as well as plastics such as nylon which can exist in the form of pellets or also as resin matrices are possible as well.

Therefore, a further subject matter of the present invention is a method for manufacturing an array fixed to a carrier material for analysis in connection with characterization, classification, differentiation, grading, staging, treatment and diagnosis of oligodendrogliomas, astrocytomas and oligoastrocytomas, in which method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the characterization, classification, differentiation, grading, staging, treatment and diagnosis of oligodendrogliomas, astrocytomas and oligoastrocytomas, which contains at least one nucleic acid according to the present invention. DNA chips are known, for example, in U.S. Pat. No. 5,837,832. Moreover, a subject matter of the present invention is a kit which may be composed, for example, of a bisulfite-containing reagent, a set of primer oligonucleotides containing at least two oligonucleotides whose sequences in each case correspond or are complementary to an 16 base long segment of the base sequences specified in the appendix (Seq. ID No.1 through Seq. ID No.120), oligonucleotides and/or PNA-oligomers as well as instructions for carrying out and evaluating the described method. However, a kit along the lines of the present invention can also contain only part of the aforementioned components.

The present invention also makes available a method for the characterization, classification, differentiation, grading, staging, treatment and diagnosis of oligodendrogliomas, astrocytomas and oligoastrocytomas, by ascertaining genetic and/or epigenetic parameters of genomic DNA by analyzing cytosine methylations and single nucleotide polymorphisms, including the following steps:

In the first step of the method the genomic DNA sample must be isolated from tissue or cellular sources. Such sources may include cell lines, histological slides, body fluids, for example cerebrospinal fluid or lymphatic fluid, or tissue embedded in paraffin; for example, brain, central nervous system or lymphatic tissue. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the chemical treatment, this may be any means standard in the state of the art, in particular with restriction endonucleases.

The genomic DNA sample is then chemically treated in such a manner that cytosine bases which are unmethylated at the 5′-position are converted to uracil, thymine, or another base which is dissimilar to cytosine in terms of hybridization behavior. This will be understood as ‘chemical pretreatment’ hereinafter.

The above described treatment of genomic DNA is preferably carried out with bisulfite (sulfite, disulfite) and subsequent alkaline hydrolysis which results in the conversion of non-methylated cytosine nucleobases to uracil or to another base which is dissimilar to cytosine in terms of base pairing behavior.

Fragments of the chemically pretreated DNA are amplified, using sets of primer oligonucleotides according to the present invention, and a, preferably heat-stable polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

In a preferred embodiment of the method, the set of primer oligonucleotides includes at least two oligonucleotides whose sequences are each reverse complementary or identical to an at least 18 base-pair long segment of the base sequences specified in the appendix (Seq. ID No.1 through Seq. ID No.120). The primer oligonucleotides are preferably characterized in that they do not contain any CpG dinucleotides. In a particularly preferred embodiment of the method, the sequence of said primer oligonucleotides are designed so as to selectively anneal to and amplify, only the oligoastrocytoma and/or brain tissue specific DNA of interest, thereby minimizing the amplification of background or non relevant DNA. In the context of the present invention, background DNA is taken to mean genomic DNA which does not have a relevant tissue specific methylation pattern, in this case the relevant tissue being brain tissue, more specifically oligodendrocytes, oligodendroglioma, astrocyte, astrocytoma or oligoastrocytoma tissue. Examples of such primers, used in the examples are contained in Table 1.

According to the present invention, it is preferred that at least one primer oligonucleotide is bonded to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminum, steel, iron, copper, nickel, silver, or gold, it being possible for other materials such as nitrocellulose or plastics to be used as well.

The fragments obtained by means of the amplification can carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualized by means of matrix assisted laser desorption/ionization mass spectrometry (MALDI) or using electron spray mass spectrometry (ESI).

The amplificates obtained in the second step of the method are subsequently hybridized to an array or a set of oligonucleotides and/or PNA probes. In this context, the hybridization takes place in the manner described in the following. The set of probes used during the hybridization is preferably composed of at least 10 oligonucleotides or PNA-oligomers. In the process, the amplificates serve as probes which hybridize to oligonucleotides previously bonded to a solid phase. The non-hybridized fragments are subsequently removed. Said oligonucleotides contain at least one base sequence having a length of 13 nucleotides which is reverse complementary or identical to a segment of the base sequences specified in the appendix, the segment containing at least one CpG dinucleotide. The cytosine of the CpG dinucleotide is the 5^{th} to 9^{th} nucleotide from the 5′-end of the 13-mer. One oligonucleotide exists for each CpG dinucleotide. Said PNA-oligomers contain at least one base sequence having a length of 9 nucleotides which is reverse complementary or identical to a segment of the base sequences specified in the appendix, the segment containing at least one CpG dinucleotide. The cytosine of the CpG dinucleotide is the 4^{th} to 6^{th} nucleotide seen from the 5′-end of the 9-mer. One oligonucleotide exists for each CpG dinucleotide.

In the fourth step of the method, the non-hybridized amplificates are removed.

In the final step of the method, the hybridized amplificates are detected. In this context, it is preferred that labels attached to the amplificates are identifiable at each position of the solid phase at which an oligonucleotide sequence is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualized by means of matrix assisted laser desorption/ionization mass spectrometry (MALDI) or using electron spray mass spectrometry (ESI). The produced fragments may have a single positive or negative net charge for better detectability in the mass spectrometer.

The aforementioned method is preferably used for ascertaining genetic and/or epigenetic parameters of genes used for the characterization, classification, differentiation, grading, staging, treatment and diagnosis of oligodendrogliomas, astrocytomas and oligoastrocytomas.

The oligomers according to the present invention or arrays thereof as well as a kit according to the present invention are intended to be used for the characterization, classification, differentiation, grading, aging, treatment and diagnosis of oligodendrogliomas, astrocytomas and oligoastrocytomas by analyzing methylation patterns of genomic DNA. According to the pre-sent invention, the method is preferably used for the analysis of important genetic and/or epigenetic parameters within genomic DNA.

The method according to the present invention is used, for example, for the characterization, classification, differentiation, grading, staging, treatment and diagnosis of oligodendrogliomas, astrocytomas and oligoastrocytomas.

The nucleic acids according to the present invention of Seq. ID No.1 through Seq. ID No.120 can be used for the characterization, classification, differentiation, grading, staging, treatment and diagnosis of oligodendrogliomas, astrocytomas and oligoastrocytomas.

The present invention moreover relates to a method for manufacturing a diagnostic reagent and/or therapeutic agent for the characterization, classification, differentiation, grading, staging, treatment and diagnosis of oligodendrogliomas, astrocytomas and oligoastrocytomas by analyzing methylation patterns of genomic DNA, the diagnostic reagent and/or therapeutic agent being characterized in that at least one nucleic acid according to the present invention (sequence IDs 1 through 120) is used for manufacturing it, preferably together with suitable additives and auxiliary agents.

A further subject matter of the present invention relates to a diagnostic reagent and/or therapeutic agent for the characterization, classification, differentiation, grading, staging, treatment and diagnosis of oligodendrogliomas, astrocytomas and oligoastrocytomas by analyzing methylation patterns of genomic DNA, the diagnostic reagent and/or therapeutic agent containing at least one nucleic acid according to the present invention (sequence IDs 1 through 120), preferably together with suitable additives and auxiliary agents.

The present invention moreover relates to the diagnosis and/or prognosis of events which are disadvantageous to patients or individuals in which important genetic and/or epigenetic parameters within their genomic DNA, said parameters obtained by means of the present invention, may be compared to another set of genetic and/or epigenetic parameters, the differences serving as the basis for a diagnosis and/or prognosis of events which are disadvantageous to patients or individuals.

In the context of the present invention the term “hybridization” is to be understood as a bond of an oligonucleotide to a completely complementary sequence along the lines of the Watson-Crick base pairings in the sample DNA, forming a duplex structure.

The term “functional variants” denotes all DNA sequences which are complementary to a DNA sequence, and which hybridize to the reference sequence under stringent conditions.

In the context of the present invention, “genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

In the context of the present invention, “epigenetic parameters” are, in particular, cytosine methylations and further chemical modifications of DNA and sequences further required for their regulation. Further epigenetic parameters include, for example, the acetylation of histones which, however, cannot be directly analyzed using the described method but which, in turn, correlates with DNA methylation.

In the context of the present invention, the term ‘treatment’ is taken to include planning of suitable methods of patient therapy (e.g. surgery, radiation therapy, chemotherapy).

In the following, the present invention will be explained in greater detail on the basis of the sequences and examples with reference to accompanying figures without being limited thereto.

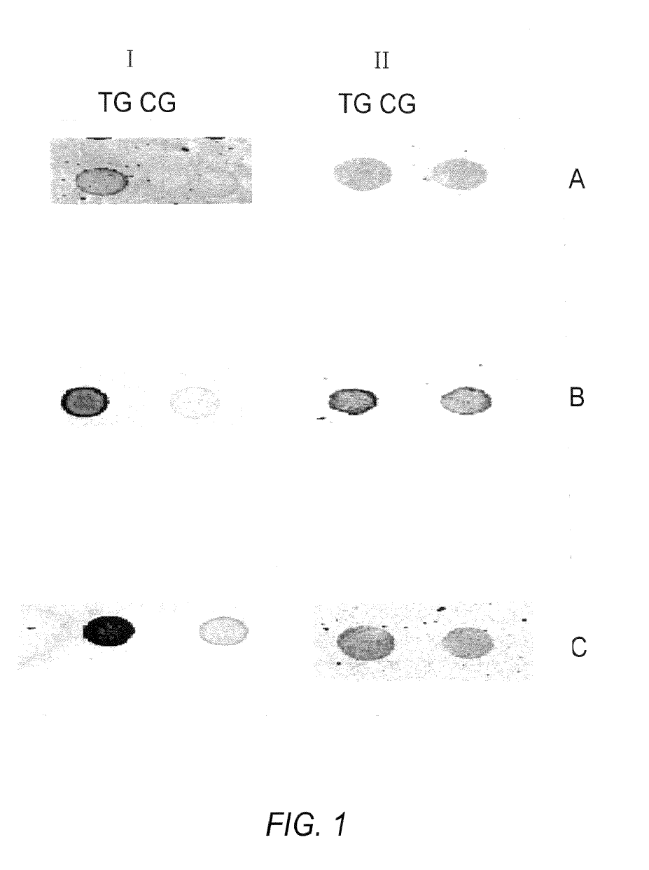

FIG. 1

FIG. 1 shows the hybridisation of fluorescent labelled amplificates to a surface bound oligonucleotide. Sample I being from oligodendroglioma (brain tumor) tissue and sample II being from oligoastrocytoma (brain tumor) tissue. Flourescence at a spot indicates hybridisation of the amplificate to the oligonucleotide. Hybridisation to a CG oligonucleotide denotes methylation at the cytosine position being analysed, hybridisation to a TG oligonucleotide denotes no methylation at the cytosine position being analysed. It can be seen that Sample I was unmethylated for CG positions (as indicated in example (1-3) of the amplificates of the genes TNF-alpha (see figure FIG. 1A), DAPK1 (see figure FIG. 1B), and WT1 (see figure FIG. 1C) whereas in comparison Sample II had a higher degree of methylation at the same position.

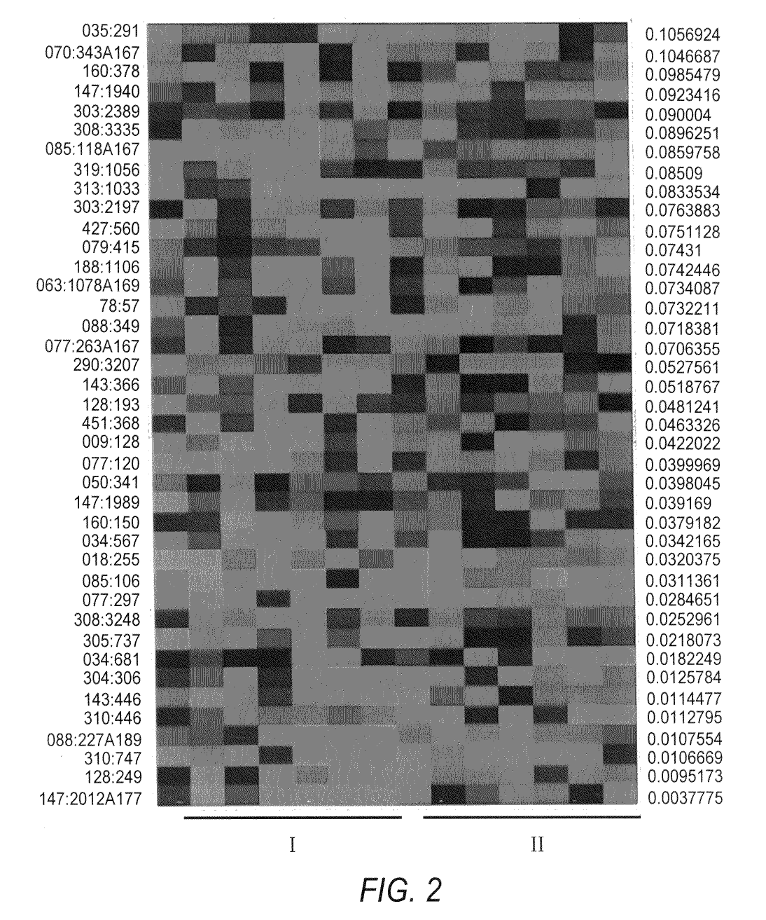

FIG. 2

Differentiation of oligodendroglyoma (1) and oligoastrocytoma (II). High probability of methylation corresponds to red, uncertainty to black and low probability to green. The labels on the left side of the plot are gene and CpG identifiers. The hybridisation was done with Cy5 labelled amplificates generated by multiplex PCR reactions as shown in Table 1. The labels on the right side give the significance (p-value, T-test) of the difference between the means of the two groups. Each row corresponds to a single CpG and each column to the methylation levels of one sample. CpGs are ordered according to their contribution to the distinction to the differential diagnosis of the two lesions with increasing contribution from top to bottom.

Seq. ID No.1 through Seq. ID No.120

Sequences having odd sequence numbers (e.g., Seq. ID No. 1, 3, 5, . . . ) exhibit in each case sequences of chemically pretreated genomic DNAs. Sequences having even sequence numbers (e.g., Seq. ID No. 2, 4, 6, . . . ) exhibit in each case the sequences of chemically pretreated genomic DNAs. Said genomic DNAs are complementary to the genomic DNAs from which the preceeding sequence was derived (e.g., the complementary sequence to the genomic DNA from which Seq. ID No.1 is derived is the genomic sequence from which Seq. ID No.2 is derived, the complementary sequence to the genomic DNA from which Seq. ID No.3 is derived is the sequence from which Seq. ID No.4 is derived, etc.) Seq. ID No.121 through Seq. ID No.132 Seq. ID No.1 through Seq. ID No.120 show sequences of oligonucleotides used in the Examples.

### EXAMPLE 1

### Methylation Analysis of the Gene DAPK1

The following example relates to a fragment of the gene DAPK1 in which a specific CG-position is to be analyzed for methylation.

In the first step, a genomic sequence is treated using bisulfite hydrogen sulfite, disulfite) in such a manner that all cytosines which are not methylated at the 5-position of the base are modified in such a manner that a different base is substituted with regard to the base pairing behavior while the cytosines methylated at the 5-position remain unchanged.

If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The chemically converted DNA is then used for the detection of methylated cytosines. In the second method step, the treated DNA sample is diluted with water or an aqueous solution. Preferably, the DNA is subsequently desulfonated. In the third step of the method, the DNA sample is amplified in a polymerase chain reaction, preferably using a heat-resistant DNA polymerase. In the present case, cytosines of the gene DAPK1 are analyzed. To this end, a defined fragment having a length of 465 bp is amplified with the specific primer oligonucleotides ATTAATATTATGTAAAGTGA (Sequence ID No. 121) and CTTACAACCATTCACCCACA (Sequence ID No. 122). The single gene PCR reaction was performed on a thermocycler (Epperdorf GmbH) using bisulfite DNA 10 ng, primer 6 pmole each, dNTP 200 μM each, 1.5 mM MgCl2 and 1 U HotstartTaq (Qiagen AG). The other conditions were as recommended by the Taq polymerase manufacturer. In the multiplex PCR up to 16 primer pairs were used within the PCR reaction. The multiplex PCR was done according the single gene PCR with the following modifications: primer 0.35 pmole each, dNTP 800 μM each and 4.5 mM MgCl2. The cycle program for single gene PCR and multiplex PCR was as followed: step 1, 14 min 96° C.; step 2, 60 sec 96° C.; step 3, 45 sec 55° C.; step 4, 75 sec 72° C.; step 5, 10 min 72° C.; the step 2 to step 4 were repeated 39 fold.

The amplificate serves as a sample which hybridizes to an oligonucleotide previously bound to a solid phase, forming a duplex structure, for example AGGAGGACGAGGTGATG (Sequence ID No. 123), the cytosine to be detected being located at position 303 of the amplificate. The detection of the hybridization product is based on Cy3 and Cy5 fluorescently labelled primer oligonucleotides which have been used for the amplification. A hybridization reaction of the amplified DNA with the oligonucleotide takes place only if a methylated cytosine was present at this location in the bisulfite-treated DNA. Thus, the methylation status of the specific cytosine to be analyzed is inferred from the hybridization product.

In order to verify the methylation status of the position, a sample of the amplificate is further hybridized to another oligonucleotide previously bonded to a solid phase. Said oligonucleotide is identical to the oligonucleotide previously used to analyze the methylation status of the sample, with the exception of the position in question. At the position to be analysed said oligonucleotide comprises a thymine base as opposed to a cytosine base i.e AGGAGGATGAGGTGATG (Sequence ID No. 124). Therefore, the hybridisation reaction only takes place if an unmethylated cytosine was present at the position to be analysed.

### EXAMPLE 2

### Methylation Analysis of the Gene TNFB

The following example relates to a fragment of the gene TNFB in which a specific CG-position is to be analyzed for methylation.

In the first step, a genomic sequence is treated using bisulfite (hydrogen sulfite, disulfite) in such a manner that all cytosines which are not methylated at the 5-position of the base are modified in such a manner that a different base is substituted with regard to the base pairing behavior while the cytosines methylated at the 5-position remain unchanged.

If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The chemically converted DNA is then used for the detection of methylated cytosines. In the second method step, the treated DNA sample is diluted with water or an aqueous solution. Preferably, the DNA is subsequently desulfonated. In the third step of the method, the DNA sample is amplified in a polymerase chain reaction, preferably using a heat-resistant DNA polymerase. In the present case, cytosines of the gene TNFB are analyzed. To this end, a defined fragment having a length of 450 bp is amplified with the specific primer oligonucleotides tttttgtttgattgaaatagtag (Sequence ID 125) and aaaaacccaaaaataaacaa (Sequence ID No. 126). The single gene PCR reaction was performed on a thermocycler (Epperdorf GmbH) using bisulfite DNA 10 ng, primer 6 pmole each, dNTP 200 μM each, 1.5 mM MgCl2 and 1 U HotstartTaq (Qiagen AG). The other conditions were as recommended by the Taq polymerase manufacturer. In the multiplex PCR up to 16 primer pairs were used within the PCR reaction. The multiplex PCR was done according the single gene PCR with the following modifications: primer 0.35 pmole each, dNTP 800 μM each and 4.5 mM MgCl2. The cycle program for single gene PCR and multiplex PCR was as followed: step 1, 14 min 96° C.; step 2, 60 sec 96° C.; step 3, 45 sec 55° C.; step 4, 75 sec 72° C.; step 5, 10 min 72° C.; the step 2 to step 4 were repeated 39 fold.

The amplificate serves as a sample which hybridizes to an oligonucleotide previously bound to a solid phase, forming a duplex structure, for example AGGGGTTTCGTATAGTAG (Sequence ID No. 127), the cytosine to be detected being located at position 149 of the amplificate. The detection of the hybridization product is based on Cy3 and Cy5 fluorescently labelled primer oligonucleotides which have been used for the amplification. A hybridization reaction of the amplified DNA with the oligonucleotide takes place only if a methylated cytosine was present at this location in the bisulfite-treated DNA. Thus, the methylation status of the specific cytosine to be analyzed is inferred from the hybridization product.

In order to verify the methylation status of the position, a sample of the amplificate is further hybridized to another oligonucleotide previously bonded to a solid phase. Said oligonucleotide is identical to the oligonucleotide previously used to analyze the methylation status of the sample, with the exception of the position in question. At the position to be analysed said oligonucleotide comprises a thymine base as opposed to a cytosine base i.e. AGGGGTTTTGTATAGTAG (Sequence ID No. 128). Therefore, the hybridisation reaction only takes place if an unmethylated cytosine was present at the position to be analysed.

### EXAMPLE 3

### Methylation Analysis of the Gene CDK4

The following example relates to a fragment of the gene CDK4 in which a specific CG-position is to be analyzed for methylation. In the first step, a genomic sequence is treated using bisulfite (hydrogen sulfite, disulfite) in such a manner that all cytosines which are not methylated at the 5-position of the base are modified in such a manner that a different base is substituted with regard to the base pairing behavior while the cytosines methylated at the 5-position remain unchanged.

If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The chemically converted DNA is then used for the detection of methylated cytosines. In the second method step, the treated DNA sample is diluted with water or an aqueous solution. Preferably, the DNA is subsequently desulfonated. In the third step of the method, the DNA sample is amplified in a polymerase chain reaction, preferably using a heat-resistant DNA polymerase. In the present case, cytosines of the gene CDK4 are analyzed. To this end, a defined fragment having a length of 474 bp is amplified with the specific primer oligonucleotides TTTTGGTAGTTGGTTATATG (Sequence ID No. 129) and AAAAATAACACAATAACTCA (Sequence ID No. 130). The single gene PCR reaction was performed on a thermocycler (Eppendorf GmbH) using bisulfite DNA 10 ng, primer 6 pmole each, dNTP 200 μM each, 1.5 mM MgCl2 and 1 U HotstartTaq (Qiagen AG). The other conditions were as recommended by the Taq polymerase manufacturer. In the multiplex PCR up to 16 primer pairs were used within the PCR reaction. The multiplex PCR was done according the single gene PCR with the following modifications: primer 0.35 pmole each, dNTP 800 μM each and 4.5 mM MgCl2. The cycle program for single gene PCR and multiplex PCR was as followed: step 1, 14 min 96° C.; step 2, 60 sec 96° C.; step 3, 45 sec 55° C.; step 4, 75 sec 72° C.; step 5, 10 min 72° C.; the step 2 to step 4 were repeated 39 fold.

The amplificate serves as a sample which hybridizes to an oligonucleotide previously bound to a solid phase, forming a duplex structure, for example GTATGGGGTCGTAGGAAT (Sequence ID No. 131), the cytosine to be detected being located at position 121 of the amplificate. The detection of the hybridization product is based on Cy3 and Cy5 fluorescently labelled primer oligonucleotides which have been used for the amplification. A hybridization reaction of the amplified DNA with the oligonucleotide takes place only if a methylated cytosine was present at this location in the bisulfite-treated DNA. Thus, the methylation status of the specific cytosine to be analyzed is inferred from the hybridization product.

In order to verify the methylation status of the position, a sample of the amplificate is further hybridized to another oligonucleotide previously bonded to a solid phase. Said oligonucleotide is identical to the oligonucleotide previously used to analyze the methylation status of the sample, with the exception of the position in question. At the position to be analysed said oligonucleotide comprises a thymine base as opposed to a cytosine base i.e GTATGGGGTTGTAGGAAT (Sequence ID No. 132). Therefore, the hybridisation reaction only takes place if an unmethylated cytosine was present at the position to be analysed.

### EXAMPLE 4

### Differentiation of Oligodendroglyoma and Oligoastrocytoma Tumours

In order to relate the methylation patterns to a specific tumour type, it is initially required to comparatively analyze the DNA methylation patterns of two groups of patients with alternative forms of a tumor, in this case one group of astrocytoma grade I and another group of astrocytoma grade II, with those of healthy tissue (FIGS. 2 A and B). These analyses were carried out, analogously to Example #. The results obtained in this manner are stored in a database and the CpG dinucleotides which are methylated differently between the two groups are identified. This can be carried out by determining individual CpG methylation rates as can be done, for example, by sequencing, which is a relatively imprecise method of quantifying methylation at a specific CpG, or else, in a very precise manner, by a methylation-sensitive “primer extension reaction”. In a particularly preferred variant, as illustrated in the preceeding examples the methylation status of hundreds or thousands of CpGs may be analysed on an oligomer array. It is also possible for the patterns to be compared, for example, by clustering analyses which can be carried out, for example, by a computer.

All clinical specimens were obtained at time of surgery, i.e. in a routine clinical situation (Santourlidis, S., Prostate 39:166-174, 1999, Florl, A. R, Br. J. Cancer 80:1312-1321, 1999). A panel of genomic fragments from 56 different genes (listed in Table 1) were bisulphite treated and amplified by 6 sets of multplex PCRs (mPCR) according to Example 1. The MPCR reactions (I,J,K,L,M,N) of the genomic, bisulphite treated DNA was done using the combination of primer pairs as indicated in Table 1. However, as will be obvious to one skilled in the art, it is also possible to use other primers that amplify the genomic, bisulphite treated DNA in an adequate manner. However the primer pairs as listed in Table 1 are particularly preferred. In order to differentiate astrocaytom grade I from healthy control samples optimal results were obtained by including at least 6 CpG dinucleotides, the most informative CpG positions for this discrimination being located within the DAPK1, TNF-alpha, WT1, cFOS and ATP5A1 genes (see figure FIG. 2A, Table 1). Most other CpGs of the panel showed different methylation patterns between the two phenotypes, too. The results prove that methylation fingerprints are capable of providing differential diagnosis of solid malignant tumours and could therefore be applied in a large number clinical situations.

TABLE 1

List of genes, reference numbers and primer oligonucleotides
according to Examples 1-4 and FIGS. 1-2.

| GENE | MPCR | | | |
|---|---|---|---|---|
| ID | SET | GENE | PCR PRIMER | PCR PRIMER |
| 81 | N | ADCYAP1 | GGTGGATTTATGGTTATTTTG | TCCCTCCCTTACCCTTCAAC |
| 292 | K | AFP | AGGTTTATTGAATATTTAGG | AACATATTTCCACAACATCC |
| 85 | L | ANT1 | GTTTAAGGTTGTTTGTGTTATAAAT | CCTCCTCCCAACTACAAAA |
| 48 | L | APOA1 | GTTGGTGGTGGGGGAGGTAG | ACAACCAAAATCTAAACTAA |
| 50 | N | APOC2 | ATGAGTAGAAGAGGTGATAT | CCCTAAATCCCTTTCTTACC |
| 87 | K | AR | GTAGTAGTAGTAGTAAGAGA | ACCCCCTAAATAATTATCCT |
| 1143 | L | ATP5A1 | AGTTTGTTTTAATTTATTGATAGGA | AACAACATCTTTACAATTACTCC |
| 1011 | L | CABL | GGTTGGGAGATTTAATTTTATT | ACCAATCCAAACTTTTCCTT |
| 77 | L | CD1A | ATTATGGTTGGAATTGTAAT | ACAAAAACAACAAACACCCC |
| 1079 | L | CD63 | TGGGAGATATTTAGGATGTGA | CTCACCTAAACTTCCCAAA |
| 99 | M | CDC25A | AGAAGTTGTTTATTGATTGG | AAAATTAAATCCAAACAAAC |
| 187 | L | CDH3 | GTTTAGAAGTTTAAGATTAG | CAAAAACTCAACCTCTATCT |
| 88 | K | CDK4 | TTTTGGTAGTTGGTTATATG | AAAAATAACACAATAACTCA |
| 310 | I | CFOS | TTTTGAGTTTTAGAATTGTTTTTAG | AAAAACCCCCTACTCATCTACTA |
| 1034 | L | CMYC | TTTTGTGTGGAGGGTAGTTG | CCCCAAATAAACAAAATAACC |
| 312 | K | CMYC | TTGTTTTTGTGGAAAAGAGG | TTTCAATCTCAAAACTCAACC |
| 313 | I | CMYC | AAAGGTTTGGAGGTAGGAGT | TTCCTTTCCAAATCCTCTTT |
| 37 | M | CRIP1 | TTTAGGTTTAGGGTTTAGTT | CCACTCCAAAACTAATATCA |
| 70 | N | CSF1 | TAGGGTTTGGAGGGAAAG | AAAAATCACCCTAACCAAAC |
| 78 | M | CSNK2B | GGGGAAATGGAGAAGTGTAA | CTACCAATCCCAAAATAACC |
| 272 | N | CTLA4 | TTTTTATGGAGAGTAGTTGG | TAACTTTACTCACCAATTAC |
| 287 | K | DAD1 | TTTTGTTGTTAGAGTAATTG | ACCTCAATTTCCCCATTCAC |
| 147 | I | DAPK1 | ATTAATATTATGTAAAGTGA | CTTACAACCATTCACCCACA |
| 319 | J | E-CADHERIN | GGGTGAAAGAGTGAGTTTTATTT | ACTCCAAAAACCCATAACTAA |
| 63 | M | EGFR | GGTGTTTGATAAGATTTGAAG | CCCTTACCTTTCTTTTCCT |
| 311 | I | EGFR | GGGTAGTGGGATATTTAGTTTTT | CCAACACTACCCCTCTAA |
| 82 | M | EGR4 | AGGGGGATTGAGTGTTAAGT | CCCAAACATAAACACAAAAT |
| 1012 | L | ELK1 | AAGTGTTTTAGTTTTTAATGGGTA | CAAACCCAAAACTCACCTAT |
| 307 | J | ERBB2 | GAGTGATATTTTTATTTTATGTTTGG | AAAACCCTAACTCAACTACTCAC |
| 308 | K | ERBB2 | GAGTTTGGGAGTTTAAGATTAGT | TCAACTTCACAACTTCATTCTTAT |
| 130 | N | GP1B | GGTGATAGGAGAATAATGTTGG | TCTCCCAACTACAACCAAAC |
| 290.2 | M | HEAT | AGAGGAGATATTTTTTATGG | AAAAATCCTACAACAACTTC |
| | | SHOCK | | |
| 290.3 | J | HEAT | AAGGATAATAATTTGTTGGG | CTTAAATACAAACTTAATCC |
| | | SHOCK | | |
| 89 | I | HUMOS | TTTATTGATTGGGAGTAGGT | CTAATTTTACAAACATCCTA |
| 1083 | N | IL13 | TTTTTAGGGTAGGGGTTGT | CCTTATCCCCCATAACCA |
| 1010 | L | LMYC | AGGTTTGGGTTATTGAGTTT | CATTATTTCCTAACTACCTTATATCTC |
| 291 | L | MC2R | ATATTTGATATGTTGGGTAG | ACCTACTACAAAAAATCATC |
| 314.2 | I | MGMT | AAGGTTTTAGGGAAGAGTGTTT | ACTCCCAATACCTCACAATATAAC |
| 427 | K | MHC | GGGTATTAGGAATTTATGTG | CAAAACACCTTCCTAACTCA |
| 401 | I | MHC | TTGTTGTTTTTAGGGGTTTTGG | TCCTTCCCATTCTCCAAATATC |
| 458 | M | MHC | AAGAGTGAGAAGTAGAGGGTT | CTACTCTCTAAAACTCCAAAC |
| 487 | M | MHC | GAGGTTAAAGGAAGTTTTGGA | AAACTAAATTCTCCCAATACC |
| 465 | L | MHC | ATTGATAGGTAGTTAGATTGG | AAAAAACTCTCATAAATCTCA |
| 451 | M | MHC | AGGAGGAAGGGTTAATAAAGA | ATCTTCCTACTACTATCTCTAAC |
| 441 | M | MHC | AGGTTGGATTTTGGGTAGGT | TCTCCTACTCTCCTAATCTC |
| 160 | M | MLH1 | TTTAAGGTAAGAGAATAGGT | TTAACCCTACTCTTATAACC |
| 94 | N | N33 | TGGAGGAGATATTGTTTTGT | TTTTTCAAATCAAAACCCTACT |
| 302 | J | NF1 | TTGGGAGAAAGGTTAGTTTT | ATACAAACTCCCAATATTCC |
| 1009 | L | NMYC | GGAGGAGTATATTTTGGGTTT | ACAAACCCTACTCCTTACCTC |
| 1018 | N | NUC | AAGTTGTGTTTTTAAAAGGGTTA | AAAAACTAAACCTACCCAATAA |
| 1007 | N | OAT | TGGAGGTGGATTTAGAGGTA | ACCAAAACCCCAAAACAA |
| 304 | J | P16 | AGGGGTTGGTTGGTTATTAG | TAATTCCAATTCCCCTACAA |
| 305 | J | P53 | GTGATAAGGGTTGTGAAGGA | CAAAAACTTACCCAATCCAA |
| 1069 | N | POMC | AGTTTTTAAATAATGGGGAAAT | ACTCTTCTTCCCCTCCTTC |
| 177 | N | PRG | AGTTGAAGTTATAAGGGGTG | AATAAAAACTCTCAAAAACC |
| 26 | K | SOD1 | AGGGGAAGAAAAGGTAAGTT | CCCACTCTAACCCCAAACCA |
| 303 | I | TGF-A | GGTTTGTTTGGGAGGTAAG | CCCCCTAAAAACACAAAA |
| 301 | J | TGF-B1 | GGGGAGTAATATGGATTTGG | CCTTTACTAAACACCTCCCATA |
| 317 | I | TIMP3 | GGTAAGGGTTTTGTGTTGTTT | CCCCCTCAAACCAATAAC |
| 128 | N | TNFB | TTTTTGTTTTTGATTGAAATAGTAG | AAAAACCCCAAAATAAACAA |
| 35 | L | UBB | TTAAGTTATTTTAGGTGGAGTTTA | ACCAAAATCCTACCAATCAC |
| 1140 | N | UNG | GTTGGGGTGTTTGAGGAA | CCTCTCCCCTCTAATTAAACA |
| 300 | J | VEGF | TGGGTAATTTTTAGGTTGTGA | CCCCAAAAACAAATCACTC |
| 188 | K | WT1 | AAAGGGAAATTAAGTGTTGT | TAACTACCCTCAACTTCCC |

1. A nucleic acid comprising a sequence at least 18 bases in length of a segment of chemically pretreated genomic DNA according to one of the sequences taken from the group of Seq. ID No.1 to Seq. ID No.120 and sequences complementary thereto.

2. An oligomer, in particular an oligonucleotide or peptide nucleic acid (PNA)-oligomer, said oligomer comprising in each case at least one base sequence having a length of at least 9 nucleotides which hybridizes to or is identical to a chemically pretreated genomic DNA according to one of the Seq ID Nos 1 to 120 according to claim 1, and sequences complementary thereto.

3. The oligomer as recited in claim 2, wherein the base sequence includes at least one CpG dinucleotide.

4. The oligomer as recited in claim 2 characterized in that the cytosine of the CpG dinucleotide is located approximately in the middle third of the oligomer.

5. A set of oligomers, comprising at least two oligomers according to any of claims 2 to 4.

6. A set of oligomers as recited in claim 5, comprising oligomers for detecting the methylation state of all CpG dinucleotides within one of the sequences according to Seq. ID Nos. 1 through 120 according to claim 1, and sequences complementary thereto.

7. A set of at least two oligonucleotides as recited in claim 2, which can be used as primer oligonucleotides for the amplification of DNA sequences of one of Seq. ID 1 through Seq. ID 120 and sequences complementary thereto, and sequences complementary thereto and segments thereof.

8. A set of oligonucleotides as recited in claim 7, characterized in that at least one oligonucleotide is bound to a solid phase.

9. Use of a set of oligomer probes comprising at least ten of the oligomers according to any of claims 5 through 8 for detecting the cytosine methylation state and/or single nucleotide polymorphisms (SNPs) in a chemically pretreated genomic DNA according to claim 1.

10. A method for manufacturing an arrangement of different oligomers (array) fixed to a carrier material for analyzing diseases associated with the methylation state of the CpG dinucleotides of one of the Seq. ID 1 through Seq. ID 120 and sequences complementary thereto, wherein at least one oligomer according to any of the claims 2 through 4 is coupled to a solid phase.

11. An arrangement of different oligomers (array) obtainable according to claim 10.

12. An array of different oligonucleotide- and/or PNA-oligomer sequences as recited in claim 11, characterized in that these are arranged on a plane solid phase in the form of a rectangular or hexagonal lattice.

13. The array as recited in any of the claims 11 or 12, characterized in that the solid phase surface is composed of silicon, glass, polystyrene, aluminium, steel, iron, copper, nickel, silver, or gold.

14. A DNA- and/or PNA-array for analyzing diseases associated with the methylation state of genes, comprising at least one nucleic acid according to one of the preceeding claims.

15. A method for characterizing, classifying and/or differentiating oligodendroglioma, astrocytoma and oligoastrocytoma tumors, characterized in that the following steps are carried out:
obtaining a biological sample containing genomic DNA,
extracting the genomic DNA,
in a genomic DNA sample, cytosine bases which are unmethylated at the 5-position are converted, by chemical treatment, to uracil or another base which is dissimilar to cytosine in terms of hybridization behavior,
fragments of the chemically pretreated genomic DNA are amplified using sets of primer oligonucleotides according to claim 7 or 8 and a polymerase, the amplificates carrying a detectable label;
amplificates are hybridized to a set of oligonucleotides and/or PNA probes according to the claims 5 and 6, or else to an array according to one of the claims 12 through 15;
the hybridized amplificates are subsequently detected.

16. A method for characterizing, classifying and/or differentiating oligodendroglioma, astrocytoma and oligoastrocytoma tumors, characterized in that the following steps are carried out:
obtaining a biological sample containing genomic DNA
extracting the genomic DNA
in a genomic DNA sample, cytosine bases which are unmethylated at the 5-position are converted, by chemical treatment, to uracil or another base which is dissimilar to cytosine in terms of hybridization behavior;
fragments of the chemically pretreated genomic DNA are amplified using sets of primer oligonucleotides according to claim 7 or 8 and a polymerase;
analysis of the sequence of the amplified DNA and determination of the methylation status of one or more cytosine positions within the genomic sample, prior to chemical treatment by reference to one or more data sets.

17. The method as recited in claims 15 or 16, characterized in that the amplification step preferentially amplifies DNA which is of particularly interest in oligodendroglioma, astrocytoma and/or oligoastrocytoma cells, based on the specific genomic methylation status of oligodendroglioma, astrocytoma and/or oligoastrocytoma cells, as opposed to background DNA.

18. The method as recited in claims 15 through 17, characterized in that the chemical treatment is carried out by means of a solution of a bisulfite, hydrogen sulfite or disulfite.

19. The method as recited in one of the claims 15 through 18, characterized in that more than ten different fragments having a length of 100-2000 base pairs are amplified.

20. The method as recited in one of the claims 15 through 19, characterized in that the amplification of several DNA segments is carried out in one reaction vessel.

21. The method as recited in one of the claims 15 through 20, characterized in that the polymerase is a heat-resistant DNA polymerase.

22. The method as recited in claim 21, characterized in that the amplification is carried out by means of the polymerase chain reaction (PCR).

23. The method as recited in one of the claims 15 through 22, characterized in that the labels of the amplificates are fluorescence labels.

24. The method as recited in one of the claims 15 through 22, characterized in that the labels of the amplificates are radionuclides.

25. The method as recited in one of the claims 15 through 22, characterized in that the labels of the amplificates are detachable molecule fragments having a typical mass which are detected in a mass spectrometer.

26. The method as recited in one of the claims 15 through 22, characterized in that the amplificates or fragments of the amplificates are detected in the mass spectrometer.

27. The method as recited in one of the claims 25 and/or 26, characterized in that the produced fragments have a single positive or negative net charge for better detectability in the mass spectrometer

28. The method as recited in one of the claims 25 through 27, characterized in that detection is carried out and visualized by means of matrix assisted laser desorption/ionization mass spectrometry (MALDI) or using electron spray mass spectrometry (ESI).

29. The method as recited in one of the claims 15 through 28, characterized in that the genomic DNA is obtained from cells or cellular components which contain DNA, sources of DNA comprising, for example, cell lines, histological slides, biopsies, cerebrospinal fluid, lymphatic fluid, tissue embedded in paraffin; for example, brain or lymphatic tissue and all possible combinations thereof.

30. A kit comprising a bisulfite (=disulfite, hydrogen sulfite) reagent as well as oligonucleotides and/or PNA-oligomers according to one of the claims 2 through 4.

31. The use of a nucleic acid according to claim 1, of an oligonucleotide or PNA-oligomer according to one of the claims 2 through 4, of a kit according to claim 30, of an array according to one of the claims 11 through 14, of a set of oligonucleotides according to one of claims 5 through 8 for the treatment of tumors and cancer, in particular gliomas, astrocytomas and oligodendromas.

32. The use of a nucleic acid according to claim 1, of an oligonucleotide or PNA-oligomer according to one of the claims 2 through 4, of a kit according to claim 30, of an array according to one of the claims 11 through 14, of a set of oligonucleotides according to one of claims 5 through 8 for the therapy of tumours and cancer, in particular gliomas, astrocytomas and oligodendromas.
